(19) United States
(12) Patent Application Publication
(10) Pub. No.: US 2024/0173221 A1
(43) Pub. Date: May 30, 2024

(54) Oral Care Compositions

(71) Applicant: Colgate-Palmolive Company, New York, NY (US)

(72) Inventors: Diana Liceth HENAO ADLEVANKIN, Martinsville, NJ (US); Gokul GOVINDARAJU, Somerville, NJ (US); Courtney Nicole HARRIS, Piscataway, NJ (US)

(73) Assignee: Colgate-Palmolive Company, New York, NY (US)

(21) Appl. No.: 18/517,201

(22) Filed: Nov. 22, 2023

Related U.S. Application Data
(60) Provisional application No. 63/427,135, filed on Nov. 22, 2022.
(60) Provisional application No. 63/427,129, filed on Nov. 22, 2022.
(60) Provisional application No. 63/427,124, filed on Nov. 22, 2022.

(51) Int. Cl. A61K 8/21; A61K 8/24; A61K 8/46; A61K 8/73; A61Q 11/00

(52) U.S. Cl. CPC A61K 8/21; A61K 8/24; A61K 8/466; A61K 8/735; A61Q 11/00

(57) ABSTRACT

This application relates to novel aqueous oral care compositions useful for combining and delivering incompatible stannous fluoride or stannous chloride and potassium salts in a high-water composition, for example, to provide effective caries prevention, protection against dental erosion, and relief from dental hypersensitivity. The compositions comprise stannous fluoride or stannous chloride, nitric acid or a water-soluble nitrate salt, a water-soluble alkali metal polyphosphate, a taurate surfactant (e.g., sodium methyl cocoyl taurate) and more than 10% water, by weight of the composition.

### CROSS-REFERENCE TO RELATED PATENT APPLICATIONS

This application claims priority to U.S. Appl. No. 63/427,129, titled Oral Care Compositions and filed Nov. 22, 2022; U.S. Appl. No. 63/427,124, titled Oral Care Compositions and filed Nov. 22, 2022; and U.S. Appl. No. 63/427,135, titled Oral Care Compositions and filed Nov. 22, 2022, the content for all of which is incorporated herein by reference in its entirety for all purposes.

### BACKGROUND

This application relates to novel aqueous oral care compositions useful for combining and delivering incompatible stannous fluoride or stannous chloride and potassium salts in a high-water composition, for example, to provide effective caries prevention, protection against dental erosion, and relief from dental hypersensitivity.

Dental plaque is a sticky biofilm or mass of bacteria that is commonly found between the teeth, along the gum line, and below the gum line margins. Dental plaque can give rise to dental caries and periodontal problems such as gingivitis and periodontitis. Dental caries tooth decay or tooth demineralization caused by acid produced from the bacterial degradation of fermentable sugar.

Oral care compositions which contain stannous ion sources exhibit excellent clinical benefits, particularly in the reduction of gingivitis. Stannous ion sources, such as stannous fluoride and stannous chloride, are well known for use in clinical dentistry with a history of therapeutic benefits over forty years. However, until recently, its popularity has been limited by its instability in aqueous solutions. The instability of stannous salts in water is primarily due to the reactivity of the stannous ion (Sn²⁺). Stannous salts readily hydrolyze at a pH above 4, resulting in precipitation from solution. It has traditionally been thought that this formation of insoluble stannous salts results in a loss of therapeutic properties.

One common way to overcome the stability problems associated with stannous ions is to limit the amount of water in the composition to very low levels, or to use a dual phase system. Both of these solutions to the stannous ion problem have drawbacks. Low water oral care compositions can be difficult to formulate with desired rheological properties, and dual-phase compositions are considerably more expensive to manufacture and package. Thus, it is preferable to formulate a high-water composition which uses an alternative means to maintain stable efficacious stannous ion concentrations.

Dentinal hypersensitivity (i.e. sensitivity) is a painful condition resulting from the movement of liquid in exposed dentin tubules from external stimuli such as pressure and temperature.

In many regions of the world, consumers prefer high foaming toothpastes over products that have a poor foaming ability. Conventional levels of surfactants usually yield acceptable foam volumes when tested in pure water. However, when the testing is performed in solutions mimicking the composition of human saliva it is hard to achieve foam levels that exceed a certain volume as human saliva has foam breaking properties.

Formulation of oral care compositions comprising stannous fluoride or stannous chloride and potassium salts is particularly challenging because of stability issues between these two ingredients. Gambogi et al., U.S. Pat. No. 6,464,963 (Colgate-Palmolive Co.), discloses that attempts to include both stannous fluoride and potassium salts, such as potassium nitrate, in a single-phase, desensitizing dental composition is hampered by the formation of insoluble stannic salts and compounds such as Sn(OH)₂ and SnO₂. Gambogi solves this problem by resorting to dual-component compositions in which one component comprises the potassium salt, along with sodium hydroxide to adjust the pH of the component to 8 to 11, and the second component comprises the stannous fluoride or other stannous salts. Other suggestions in the prior art for formulations combining fluoride and stannous salts similarly call for very low water contents, e.g., less than 10% water.

It has also been reported that aqueous oral care compositions comprising unstabilized stannous ion and nitrate ion together may form potentially toxic species such as nitrite ion and nitrosamines, due to the reduction of the nitrate ion by the stannous ion. See, e.g., Strand, US 2011/0020247 (Procter & Gamble Co.); Heckendorn et al., U.S. Pat. No. 8,926,950 (GABA Int'l Holding). Strand also reports the further difficulty that fluoride ions in an oral care composition tend to precipitate out of solution when potassium nitrate is present, due to the low solubility of ionic fluoride sources.

While it has been generally suggested that oral care compositions comprising stannous salts, fluoride salts, and polyphosphate could be prepared, many references do not take issue with or seem to be aware of the unique formulation difficulties which may be encountered. See, e.g., Yu et al., U.S. Pat. No. 5,811,079 (Warner-Lambert Co.); Midha et al., US 2017/0281486 (Procter & Gamble Co.). Other reference disclosing similar compositions avoid the issues by resorting to dual-component manufactures.

Another ingredient that can be found in oral care compositions is sodium lauryl sulfate. Sodium lauryl sulfate can be useful as it may increase phase stability (e.g., reduces the likelihood of phase separation) of the oral care composition and generally does not impact the stability of the active ingredients in the oral care composition. In some cases, a surfactant substitution-adding another surfactant to replace sodium lauryl sulfate—has a negative impact on the taste or stability of the active ingredients. Moreover, microbiological stability of the formulation can be negatively impacted by the absence of sodium lauryl sulfate. There are also production benefits to having sodium lauryl sulfate in a given formulation. For example, by replacing sodium lauryl sulfate with other surfactants, the oral care composition may become overly aerated during production and it may be more difficult to clean the equipment after the production process

There is an ongoing desire for improved oral care compositions.

### BRIEF SUMMARY

The disclosure provides a combination of stannous fluoride or stannous chloride, nitric acid or a soluble nitrate salt, an effective amount of a taurate surfactant (e.g., sodium methyl cocoyl taurate), and an alkali metal polyphosphate salt in high-water oral care composition that results in stability of stannous, fluoride and nitrate. Preferably, the nitrate salt is an alkali metal nitrate (e.g., potassium nitrate) and the alkali metal polyphosphate is a pyrophosphate, tripolyphosphate, tetraphosphate or hexametaphosphate (e.g., sodium or potassium pyrophosphate). In some embodiments, the composition comprises at least 10% water, e.g., at least 20%, at least 50%, or at least 75% w/w of water. In some embodiments, the composition has a pH above 6.0, e.g., of about 7.

The disclosure further provides methods of stabilizing stannous ion in an aqueous oral care composition formulating the composition with a nitrate ion source (e.g., potassium nitrate) and a polyphosphate ion source (e.g., sodium or potassium pyrophosphate) in a high-water composition (e.g., at least 10% w/w of water), optionally wherein the solution has a pH above 6.0 (e.g., about 7).

It is also surprisingly found that the stabilization of stannous using nitrate and polyphosphate according to the present disclosure can result in clear and translucent toothpaste and gel compositions.

The disclosure further provides single-component oral care composition packages comprising the compositions disclosed herein.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

The following description of the preferred embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight relative to the total composition. The amounts given are based on the active weight of the material.

As is usual in the art, the compositions described herein are sometimes described in terms of their ingredients, notwithstanding that the ingredients may disassociate, associate or react in the formulation. Ions, for example, are commonly provided to a formulation in the form of a salt, which may dissolve and disassociate in aqueous solution. It is understood that the invention encompasses both the mixture of described ingredients and the product thus obtained.

In a first aspect, the present disclosure provides an oral care composition (Composition 1) having a single phase, wherein the oral care composition comprises:
(i) stannous fluoride or stannous chloride;
(ii) nitric acid or a water-soluble nitrate salt (e.g., potassium nitrate);
(iii) a water-soluble alkali metal polyphosphate (e.g., sodium or potassium pyrophosphate or tripolyphosphate);
(iv) an effective amount of a taurate surfactant represented by Formula (1):

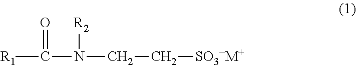

wherein R₁ is a saturated or unsaturated, straight or branched alkyl chain with 6 to 18 C atoms R₂ is H or methyl, and M⁺ is H, sodium, or potassium (e.g., wherein the taurate surfactant is sodium methyl cocoyl taurate); and
(v) more than 10% water, by weight of the composition.

For example, the disclosure provides embodiments of Composition 1 as follows:
1.1 Composition 1, wherein the water-soluble nitrate salt is selected from an alkali or alkaline earth metal nitrate, or zinc nitrate, silver nitrate, or ammonium nitrate.
1.2 Composition 1.1, wherein the water-soluble nitrate salt is an alkali metal nitrate salt or an alkaline earth metal nitrate salt.
1.3 Composition 1.2, wherein the nitrate salt is selected from lithium nitrate, sodium nitrate, potassium nitrate, magnesium nitrate, and calcium nitrate.
1.4 Composition 1.3, wherein the nitrate salt is potassium nitrate.
1.5 Any foregoing composition, wherein the water-soluble alkali metal polyphosphate is selected from a pyrophosphate, tripolyphosphate, tetraphosphate or hexametaphosphate.
1.6 Any foregoing composition, wherein the water-soluble alkali metal polyphosphate is a sodium or potassium polyphosphate.
1.7 Any foregoing composition, wherein the water-soluble alkali metal polyphosphate is selected from sodium pyrophosphate, potassium pyrophosphate, sodium tripolyphosphate and potassium tripolyphosphate.
1.8 Composition 1.7, wherein the sodium pyrophosphate salt is selected from sodium acid pyrophosphate (i.e., disodium pyrophosphate) and tetrasodium pyrophosphate.
1.9 Any foregoing composition, wherein the water-soluble nitrate salt is potassium nitrate and the water-soluble alkali metal polyphosphate salt is tetrasodium pyrophosphate.
1.10 Any foregoing composition, wherein the composition comprises a molar ratio of alkali metal polyphosphate (e.g., tetrasodium pyrophosphate) to stannous fluoride or stannous chloride of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.11 Any foregoing composition, wherein the composition comprises a molar ratio of nitric acid or water-soluble nitrate salt (e.g., potassium nitrate) to stannous fluoride or stannous chloride of at least 0.3:1, e.g., 0.3:1 to 20:1, or 0.5:1 to 20:1, or 1:1 to 20:1, or 1:1 to 15:1, or 1:1 to 10:1, or 1:1 to 5:1 or 1:1 to 3:1, or about 1:1.
1.12 Any foregoing composition, wherein the composition comprises from 0.1 to 2% stannous fluoride or stannous chloride, by weight of the composition, e.g., 0.1 to 1%, or 0.25 to 0.75%, or about 0.45%.
1.13 Any foregoing composition, wherein the composition comprises from 0.1 to 5% of the nitric acid or water-soluble nitrate salt (e.g., potassium nitrate), by weight of the composition, e.g., 0.1 to 2%, or 0.1 to 1%, or 0.1 to 0.5%, or 0.2 to 0.4%, or 0.25 to 0.75%, about 0.3%, or about 0.5%.
1.14 Any foregoing composition, wherein the composition comprises from 0.1 to 5% of the alkali metal polyphosphate salt (e.g., tetrasodium pyrophosphate or sodium tripolyphosphate), by weight of the composition, e.g., 0.8 to 5%, or 0.8 to 4%, or 0.8 to 3%, or 0.8 to 2%, or 0.8 to 1.0%, or 1.0 to 1.5%, or about 0.8%, or about 1.2%.
1.15 Any foregoing composition, wherein the composition comprises at least 10% water by weight of the composition, e.g., at least 20%, at least 30%, or at least 40%, or at least 50%, or at least 60% or at least 65%, up to 95% water, by weight of the composition, or about 20%, or about 30%, or about 40%, or about 60% or about 80%.
1.16 Any foregoing composition wherein the composition comprises 70% to 95% water, by weight of the composition, e.g., from 75% to 95%, or from 75% to 90%, or from 75% to 85%, or from 75% to 80%; or wherein the composition comprises from 10% to 50% water, by weight of the composition, e.g., 10% to 40%, or 10% to 30%, or about 20%, or about 25% by wt.
1.17 Any of the preceding oral care compositions, wherein the composition comprises water from 10%-40% by wt. (e.g., about 25% by wt.).
1.18 Any foregoing composition, wherein the composition comprises one or more humectants (e.g., glycerin, sorbitol, propylene glycol, or a mixture thereof) in a net amount of 5% to 70% by weight of the composition, e.g., from 5% to 25% by weight of the composition, or from 10% to 25%, or from 15% to 25%, or about 20%, or from 30 to 70%, or from 35 to 60%, or from 40 to 60%, or from 60 to 70%. by weight of the composition.
1.19 Any foregoing composition, wherein the composition is a single phase, i.e., it does not form two phases on standing.
1.20 Any foregoing composition, wherein the composition is dual phase, i.e., it forms two phases on standing.
1.21 Composition 1.19, wherein the composition forms an emulsion immediately upon mixing, and separates into two phases upon standing within 10 minutes (e.g., within 5 minutes, or within 3 minutes, or within 1 minute).
1.22 Any foregoing composition, wherein the composition is a clear (e.g., not opaque or turbid) solution (e.g., not a suspension) or a clear (e.g., translucent, not opaque) semisolid or gel.
1.23 Any foregoing composition, wherein the composition is physically and chemically stable, for example, wherein no color change or precipitation occurs on storage at ambient conditions for 3 months or more (e.g., 6 months or more, or 1 year or more).
1.24 Composition 1.22, wherein the stannous ion concentration is substantially stable for at least three months on storage, e.g., the concentration of stannous ion is at least 80% of the original concentration, or at least 85%, or at least 90%.
1.25 Any foregoing composition, wherein the composition has a pH of between 5 and 9, or a pH between 6 and 8, or a pH between 6.5 and 7.5, or a pH between 6.9 and 7.1, or a pH of about 7.
1.26 Any foregoing composition, wherein the composition comprises less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids.
1.27 Any foregoing composition, wherein the composition is free or substantially free of any hydrophobic liquid or mixture of hydrophobic liquids (e.g., less than 0.1% by weight of the composition).
1.28 Any of Compositions 1 or 1.1-1.24, wherein the composition comprises at least 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, 10-90% by weight, or 20-80% by weight, or 30-70% by weight, or 30-50% by weight, or 10-50% by weight, or 10-30% by weight, of such hydrophobic liquids.
1.29 Any foregoing composition, further comprising a nonionic surfactant, e.g., a hydrophilic nonionic surfactant.
1.30 Composition 1.28, wherein the nonionic surfactant is a copolymer of ethylene oxide and propylene oxide, for example, a block copolymer (e.g., a triblock copolymer).
1.31 Composition 1.29, wherein the nonionic surfactant is a poloxamer, e.g., a triblock copolymer having a hydrophobic polypropylene glycol block flanked by hydrophilic polyethylene glycol blocks.
1.32 Composition 1.30, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.33 Any foregoing composition, comprising a nonionic surfactant in an amount of 0.01 to 5.0%, by weight of the composition, e.g., 0.1 to 1.0%, 0.2 to 0.7%, 0.3 to 0.5%, about 0.4%.
1.34 Any foregoing composition wherein the composition further comprises one or more of a thickener (e.g., xanthan gum or carboxymethyl cellulose, such as sodium salt), a buffer, a sweetener, a flavorant, a pigment, a dye, an anti-caries agent, an anti-bacterial agent, a whitening agent, a desensitizing agent, a preservative, or a mixture thereof.
1.35 Any foregoing composition wherein the composition further comprises an additional fluoride ion source.
1.36 Composition 1.35, wherein the additional fluoride ion source is selected from sodium fluoride, potassium fluoride, sodium monofluorophosphate, sodium fluorosilicate, ammonium fluorosilicate, amine fluoride (e.g., N′-octadecyltrimethylendianiine-N,N,N-tris(2-ethanol)-dihydrofluoride), ammonium fluoride, titanium fluoride, hexafluorosulfate, or a mixture thereof.
1.37 Any foregoing composition wherein the composition comprises a whitening agent.
1.38 Any foregoing composition wherein the composition comprises a whitening agent, wherein the whitening agent is hydrogen peroxide.
1.39 Any foregoing composition wherein the composition is a mouthwash.
1.40 Any foregoing composition wherein the composition is a dentifrice (e.g., a toothpaste or a tooth gel).
1.41 Any foregoing composition, wherein the composition is free of abrasives (e.g., the composition is free of silicas).
1.42 Any foregoing composition, wherein the composition comprises abrasive (e.g. silicas) in an amount of 1-30;% by weight of the composition, e.g., 10-30%, or 20-25%, or 15-20%. The one or more abrasive(s) may include: silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, and combinations and derivatives thereof.
1.43 Any of the foregoing compositions, wherein the composition is effective upon application to the oral cavity, e.g., by rinsing and/or brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.
1.44 Any foregoing composition, wherein the composition has enhanced stannous ion stability (e.g., compared to a composition comprising stannous fluoride or stannous chloride without both a nitrate ion source and a polyphosphate).
1.45 Any foregoing composition, wherein the composition is packaged in a container comprising a single storage compartment, which compartment comprises the composition, and a closure (e.g., a screw-top closure) which seals the compartment.
1.46 Any foregoing composition further comprising one or more of a zwitterionic surfactant (e.g., betaine), and/or a nonionic polymer (e.g., a polyethylene glycol, such as PEG-600).
1.47 The preceding composition, wherein the nonionic polymer comprises polyethylene glycol (e.g., from about 0.5% to about 5% by wt.) (e.g., about 2% by wt. of the total weight of the oral care composition).
1.48 The composition of 1.47, wherein the oral care composition comprises a zwitterionic surfactant (e.g., cocamidopropyl betaine).
1.49 The preceding composition, wherein the zwitterionic surfactant comprises cocamidopropyl betaine (e.g., from about 0.5% to about 5% by wt. of the total weight of the oral care composition) (e.g., about 1.25% by wt.).
1.50 Any foregoing composition, wherein the composition has less than 20% by weight of any one polymeric thickener (e.g., xanthan gum, carrageenan gum, carboxymethyl cellulose, such as sodium CMC), such as less than 15% by weight, or less than 10% by weight, or less than 5% by weight, or less than 1% by weight, or 0.05-1%, or 0.05-0.5%, or 0.25 to 0.75%, by weight, or about 0.5% by weight, based on the total weight of the oral care composition.
1.51 The preceding composition, wherein the composition comprises xanthan gum (e.g., from about 0.1% to about 1% by wt. of the total oral care composition).
1.52 Any foregoing composition, wherein the composition has less than 40% by weight of any silica (e.g., thickening silica), such as 5-40%, or 5-30%, or 5-25% or about 23% by weight.
1.53 Any foregoing composition, wherein the composition is translucent or transparent. Translucent, as used herein, refers to the characteristic of an oral care composition to permit the passage of light but does not necessarily allow for detailed objects to be distinguished through a thickness of 1 cm of the oral care composition. The term “transparent” with respect to oral care compositions of the present disclosure indicates that the oral care composition has transmittance of visible light of at least 65%, for example measured using a Lambda 40 UV-visible or Lambda 25 UV-visible spectrometer. The oral care compositions may have, for example, a transmittance of at least 70%, at least 75%, at least 80%, at least 85%, at least 88%, at least 90%, or at least 95% at a wavelength of 600 nm, measured, for example, using a Lambda 40 UV-visible spectrometer or Lambda 25 UV-visible spectrometer.
1.54 Any foregoing composition, in the form of a gel having cylindrical cross section (e.g., diameter of 5-15 mm or 8-10 mm).
1.55 Any foregoing composition, in the form of a gel having a flat ribbon cross-section (e.g., with a thickness of 2-15 mm 5-10 mm).
1.56 Any of the preceding oral care compositions, wherein the R₁ is a saturated or unsaturated, straight or branched alkyl chain with 8 to 14 C atoms.
1.57 Any of the preceding oral care compositions, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, and combinations thereof.
1.58 Any of the preceding oral care compositions, wherein the taurate surfactant comprises one or more surfactant selected from the group consisting of: sodium lauroyl methyl taurate (or sodium methyl lauroyl taurate), sodium methyl cocoyl taurate, and combinations thereof.
1.59 Any of the preceding oral care compositions, wherein the taurate surfactant comprises sodium methyl cocoyl taurate (e.g., about 0.5% to about 10%, by wt., of sodium methyl cocoyl taurate based on the total weight of the oral care composition) (e.g., 1%-5% by wt. of sodium methyl cocoyl taurate) (e.g., about 2.5% by wt. sodium methyl cocoyl taurate, based on the total weight of the oral care composition).
1.60 Any of the preceding oral care compositions, wherein the taurate surfactant is present in an amount of from about 0.25% to about 5%, e.g., from about 0.4% to about 3%, e.g., from about 0.4% to about 2.75%, e.g., from about 0.4% to about 2.5%, e.g., from about 0.5% to about 3%, e.g., from about 0.8% to about 3%, e.g., from about 1% to about 3%, e.g., from about 1.2% to about 2.7%, e.g., from about
1.5% to about 3%, e.g., from about 2% to about 3%, e.g., from about 1% to about 2.8%, e.g., from about 1% to about 2.7%, e.g., from about 1% to about 2.5%, e.g., from about 1.5% to about 2.8%, e.g., from about 1.5% to about 2.5%, e.g., from about 1.8% to about 3%, e.g., from about 1.8% to about 2.8%, e.g., from about
1.8% to about 2.7%, e.g., from about 1.8% to about 2.5%, e.g., about 2.5% by weight of the oral care composition.
1.61 Any of the preceding oral care compositions, wherein the taurate surfactant comprises sodium methyl cocoyl taurate in an amount from 0.25%-5% by wt. of the total composition (e.g., about 1 by wt. of the total composition).
1.62 Any of the preceding oral care compositions, wherein the oral care composition comprises hyaluronic acid (e.g., high-molecular weight hyaluronic acid (HA)) or sodium hyaluronate (e.g., average MW>100,000 Da, e.g., 300 kDa-1 MDa).
1.63 Any preceding oral care composition, wherein the composition comprises:
   Stannous fluoride from 0.1 to 2%;
   Potassium nitrate from 0.1 to 2% by wt.;
   Tetrasodium pyrophosphate from 0.1 to 5% by wt.;
   Sodium methyl cocoyl taurate from 0.25%-5% by wt; and
   Water from 15%-30% by wt. (e.g., about 24% by wt.)
1.64 Any preceding oral care composition, wherein the composition comprises:
   Stannous fluoride from 0.1 to 2%;
   Potassium nitrate from 0.1 to 2% by wt.;
   Sodium tripolyphosphate from 0.1 to 5% by wt.;
   Sodium methyl cocoyl taurate from 0.25%-5% by wt; and
   Water from 15%-30% by wt. (e.g., about 24% by wt.)

The compositions may optionally comprise additional ingredients suitable for use in oral care compositions. The compositions of the disclosure, e.g., any of Composition 1.0 et seq or Composition 2.0 et seq, may be formulated in a suitable dentifrice base, e.g., comprising abrasives, e.g., silica abrasives, surfactants, foaming agents, vitamins, polymers, enzymes, humectants, thickeners, additional antimicrobial agents, preservatives, flavorings, colorings, and/or combinations thereof. Examples of suitable additional ingredients that can be employed in the compositions of the present disclosure are discussed in more detail below.

In a second aspect, the present disclosure further provides a method (Method 1) of stabilizing stannous ion in an aqueous oral care composition comprising the steps of (1) providing an aqueous vehicle, (2) adding to the aqueous vehicle a stannous ion source, (3) adding to the aqueous vehicle a nitrate ion source, and 4) incorporating a polyphosphate ion source and a taurate surfactant into the aqueous vehicle, wherein the final composition is a single-phase high-water composition.

For example, the disclosure provides embodiments of Method 1 as follows:
1.1 Method 1, wherein the stannous ion source is a water-soluble stannous salt.
1.2 Method 1 or 1.1, wherein the stannous salt is selected from stannous chloride and stannous fluoride.
1.3 Method 1.2, wherein the stannous salt is stannous fluoride.
1.4 Any preceding method, wherein the nitrate ion source is nitric acid or a water-soluble nitrate salt.
1.5 Method 1.4, wherein the water-soluble nitrate salt is selected from an alkali or alkaline earth metal nitrate, or zinc nitrate, silver nitrate, or ammonium nitrate.
1.6 Method 1.4, wherein the water-soluble nitrate salt is an alkali metal nitrate salt or an alkaline earth metal nitrate salt.
1.7 Method 1.6, wherein the nitrate salt is selected from lithium nitrate, sodium nitrate, potassium nitrate, magnesium nitrate, and calcium nitrate.
1.8 Method 1.7, wherein the nitrate salt is potassium nitrate.
1.9 Any preceding method, wherein the polyphosphate ion source is a water-soluble alkali metal polyphosphate.
1.10 Method 1.9, wherein the water-soluble alkali metal polyphosphate is selected from a pyrophosphate, tripolyphosphate, tetraphosphate or hexametaphosphate.
1.11 Method 1.10, wherein the water-soluble alkali metal polyphosphate is a sodium or potassium polyphosphate.
1.12 Method 1.11, wherein the water-soluble alkali metal polyphosphate is selected from sodium pyrophosphate, potassium pyrophosphate, sodium tripolyphosphate and potassium tripolyphosphate.
1.13 Method 1.12, wherein the sodium pyrophosphate salt is selected from sodium acid pyrophosphate (i.e., disodium pyrophosphate) and tetrasodium pyrophosphate.
1.14 Any preceding method, wherein the stannous salt is stannous fluoride, the nitrate salt is potassium nitrate, and the polyphosphate salt is tetrasodium pyrophosphate.
1.15 Any preceding method, wherein the composition is formulated to have a molar ratio of polyphosphate source (e.g., tetrasodium pyrophosphate or sodium tripolyphosphate) to stannous source (e.g., stannous fluoride) of at least 1:1, e.g., 1:1 to 5:1, or 1:1 to 4:1, or 1:1 to 3:1, or 1:1 to 2:1, or 1.5:1 to 5:1, or 2:1 to 5:1, or 2:1 to 4:1, or 2:1 to 3:1, or about 1:1.
1.16 Any preceding method, wherein the composition is formulated to have a molar ratio of nitric acid or nitrate source (e.g., potassium nitrate) to stannous source (e.g., stannous fluoride) of at least 0.3:1, e.g., 0.3:1 to 20:1, or 0.5:1 to 20:1, or 1:1 to 20:1, or 1:1 to 15:1, or 1:1 to 10:1, or 1:1 to 5:1 or 1:1 to 3:1, or about 1:1.
1.17 Any preceding method, wherein the composition is formulated to comprise from 0.1 to 2% stannous ion source (e.g., stannous fluoride), by weight of the composition, e.g., 0.1 to 1%, or 0.25 to 0.75%, or about 0.45%, by weight based on the total weight of the oral care composition.
1.18 Any preceding method, wherein the composition is formulated to comprise from 0.1 to 5% of nitrate ion source (e.g., potassium nitrate, nitric acid, etc.), by weight of the composition, e.g., about 0.1 to about 2%, or about 0.1 to about 1%, or about 0.1 to about 0.5%, or about 0.2 to about 0.4%, or about 0.25 to about 0.75%, about 0.3%, or about 0.5%, by weight based on the total weight of the oral care composition.
1.19 Any preceding method, wherein the composition is formulated to comprise from about 0.1 to about 5% of polyphosphate ion source (e.g., tetrasodium pyrophosphate), by weight of the composition, e.g., about 0.8 to about 5%, or about 0.8 to about 4%, or about 0.8 to about 3%, or about 0.8 to about 2%, or about 0.8 to about 1%, or about 1 to about 1.5%, or about 0.8%, or about 1.2%.
1.20 Any preceding method, wherein the aqueous vehicle comprises water and optionally one or more humectants (e.g., glycerin, sorbitol, propylene glycol, or a mixture thereof).
1.21 Any preceding method, wherein the composition is formulated to comprise from about 10% to about 95% water, by weight of the composition, e.g., from about 20 to about 95%, or from about 30 to about 95%, or from about 40 to about 95%, or from about 50 to about 95%, or from about 60 to about 95% or from about 65 to about 95%, by weight of the composition, or about 20%, or about 40%, or about 60% or about 80%.
1.22 Any preceding method, wherein the composition is formulated to comprise about 70% to about 95% r water, by weight of the composition, e.g., from about 75% to about 95%, or from about 75% to about 90%, or from about 75% to about 85%, or from about 75% to about 80%; or wherein the composition is formulated to comprise from about 10% to about 50% water, by weight of the composition, e.g., about 10% to about 40%, or about 10% to about 30%, or about 20%. In some instances, the oral care composition comprises water in an amount from about 70 to about 95 wt. %, about 70 to about 90 wt. %, about 70 to about 85 wL.%, about 70 to about 80 wt. %; from about 75 to about 95 wt. %, about 75 to about 90 wt. %, about 75 to about 85 wt. %; from about 80 to about 95 wt. %, about 80 to about 90, or any range or subrange thereof, based on the total weight of the oral care composition. In other instances, the amount of water in the oral care composition is about 10 to about 50 wt. %, about 15 to about 50 wt. %, about 20 to about 50 wt. %, about 30 to about 50 wt. %; from about 10 to about 45 wt. %, about 15 to about 45 wt. %, about 20 to about 45 wt. %, about 30 to about 45 wt. %; from about 10 to about 40 wt. %, about 15 to about 40 wt. %, about 20 to about 40 wt. %, about 30 to about 40 wt. %, or any range or subrange thereof, based on the total weight of the oral care composition.
1.23 Any preceding method, wherein the composition is formulated to comprise one or more humectants (e.g., glycerin, sorbitol, propylene glycol, or a mixture thereof) in a net amount of about 5 to about 75% by weight of the composition, e.g., from about 5% to about 25% by weight of the composition, or from about 10% to about 25%, or from about 15% to about 25%, or about 20%, or from about 30 to about 70%, or from about 35 to about 60%, or from about 40 to about 60%, or from about 60 to about 70%, by weight of the composition.
1.24 Any preceding method, wherein the composition is formulated as a clear (e.g., not opaque or turbid) solution (e.g., not a suspension) or a clear (e.g., translucent, not opaque) semisolid or gel.
1.25 Any preceding method, wherein the composition is physically and chemically stable, for example, wherein no color change or precipitation occurs on storage at ambient conditions for 3 months or more (e.g., 6 months or more, or 1 year or more).
1.26 Method 1.27, wherein the stannous ion concentration is substantially stable for at least three months on storage, e.g., the concentration of stannous ion is at least 80% of the original concentration, or at least 85%, or at least 90%.
1.27 Any preceding method, wherein the composition has a pH of between 5 and 9, or a pH between 6 and 8, or a pH between 6.5 and 7.5, or a pH between 6.9 and 7.1, or a pH of about 7.
1.28 Any preceding method, wherein the composition is formulated to comprise less than 10% of any hydrophobic liquid or mixture of hydrophobic liquids (e.g., alkyl fatty acid esters (e.g., isopropyl myristate), vegetable oils, mineral oils, or combinations thereof), by weight of the composition, for example, less than 5% by weight or less than 3% by weight or less than 1% by weight, of such hydrophobic liquids, based on the total weight of the oral care composition.
1.29 Any preceding method, wherein the composition is formulated to be free or substantially free of any hydrophobic liquid or mixture of hydrophobic liquids (e.g., less than 0.1% by weight of the oral care composition), i.e., the method does not comprise any step of adding any hydrophobic liquid to the aqueous vehicle.
1.30 Any preceding method, wherein the composition is formulated to comprise a nonionic surfactant, e.g., a hydrophilic nonionic surfactant, i.e., the method further comprises the step (5) of adding a nonionic surfactant to the aqueous vehicle.
1.31 Method 1.31, wherein the nonionic surfactant is a copolymer of ethylene oxide and propylene oxide, for example, a block copolymer (e.g., a triblock copolymer).
1.32 Method 1.31, wherein the nonionic surfactant is a poloxamer, e.g., a triblock copolymer having a hydrophobic polypropylene glycol block flanked by hydrophilic polyethylene glycol blocks.
1.33 Method 1.33, wherein the poloxamer has a polyethylene glycol block length of about 75 to 125 units (e.g., about 100-101), and a polypropylene block length of about 25 to 75 units (e.g., about 55-56), for example, poloxamer 407 or Pluronic F127.
1.34 Any of methods 1.31-1.34, wherein the composition is formulated to comprise the nonionic surfactant in an amount of 0.01 to 5.0%, by weight of the composition, e.g., 0.1 to 1.0%, 0.2 to 0.7%, 0.3 to 0.5%, about 0.4%, by weight based on the total oral care composition
1.35 Any preceding method, wherein the composition is a dentifrice (e.g., a toothpaste or a tooth gel).
1.36 Any preceding method, wherein the composition is formulated to comprise abrasive (e.g. silicas) in an amount of about 1 to about 30% by weight of the composition, e.g., about 10 to about 30%, or about 20 to about 25% by weight based on the oral care composition.
1.37 Any preceding method, wherein the composition is formulated to be free of abrasives (e.g., the composition is formulated to be free of silicas).
1.38 Any preceding method, wherein step (1) occurs first and steps (2)-(5) occur in any order.
1.39 Any preceding method, further comprising a final step (6) of packaging the composition in a container comprising a single storage compartment, which compartment comprises the composition, and a closure (e.g., a screw-top closure) which seals the compartment.
1.40 Any preceding method, wherein the method results in a composition according to Composition 1, or any of 1.1-1.64 thereof.
1.41 Any of the preceding methods, wherein the method comprises adding an effective amount of a taurate surfactant represented by Formula (1):

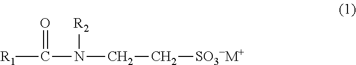

wherein R₁ is a saturated or unsaturated, straight or branched alkyl chain with 6 to 18 C atoms R₂ is H or methyl, and M⁺ is H, sodium, or potassium (e.g., wherein the taurate surfactant is sodium methyl cocoyl taurate).
1.42 The preceding method, wherein the R₁ is a saturated or unsaturated, straight or branched alkyl chain with 8 to 14 C atoms.
1.43 Any of the preceding methods, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, and a combination of two or more thereof.
1.44 Any of the preceding methods, wherein the taurate surfactant comprises one or more surfactant selected from the group consisting of: sodium lauroyl methyl taurate (or sodium methyl lauroyl taurate), sodium methyl cocoyl taurate, and combinations thereof.
1.45 Any of the preceding methods, wherein the taurate surfactant comprises sodium methyl cocoyl taurate (e.g., about 0.5% to about 10% by wt. of sodium methyl cocoyl taurate) (e.g., about 1% to about 5% by wt. of sodium methyl cocoyl taurate) (e.g., about 2.5% by wt. sodium methyl cocoyl taurate).
1.46 Any of the preceding methods, wherein the taurate surfactant is present in an amount of from about 0.25% to about 5%, e.g., from about 0.4% to about 3%, e.g., from about 0.4% to about 2.75%, e.g., from about 0.4% to about 2.5%, e.g., from about 0.5% to about 3%, e.g., from about 0.8% to about 3%, e.g., from about 1% to about 3%, e.g., from about 1.2% to about 2.7%, e.g., from about 1.5% to about 3%, e.g., from about 2% to about 3%, e.g., from about 1% to about 2.8%, e.g., from about 1% to about 2.7%, e.g., from about 1% to about 2.5%, e.g., from about 1.5% to about 2.8%, e.g., from about 1.5% to about 2.5%, e.g., from about 1.8% to about 3%, e.g., from about 1.8% to about 2.8%, e.g., from about 1.8% to about 2.7%, e.g., from about 1.8% to about 2.5%, e.g., about 2.5% by weight of the composition.
1.47 Any of the preceding methods, wherein the taurate surfactant comprises sodium methyl cocoyl taurate in an amount from about 0.25% to about 5% by wt. of the total composition (e.g., about 2.5% by wt. of the total composition).

In a third aspect, the present disclosure provides an oral care package comprising a composition according to Composition 1, or any of 1.1-1.66. The oral care package may comprises a container comprising a single storage compartment, which compartment contains the composition, and a closure (e.g., a screw-top closure) which seals the compartment. In some embodiments, wherein the composition is a toothpaste or gel, the package comprises a closure which dispenses a ribbon of toothpaste or gel having a circular cross-section, oval cross-section, or flat-ribbon cross-section. In some embodiments, such ribbon is dispensed having a diameter or thickness of 5-25 mm, e.g., 5-10 mm, or 10-15 mm, or 15-20 mm, or 20-25 mm.

In a fourth aspect, the present disclosure provides a method of treatment or prevention of gingivitis, plaque, dental caries, and/or dental hypersensitivity, the method comprising the application to the oral cavity of a person in need thereof, of a composition according to the invention (e.g., Composition 1 et seq.), e.g., by brushing, for example, one or more times per day.

Alternatively, the present disclosure provides Composition 1, et seq., for use in the treatment or prevention of gingivitis, plaque, dental caries, and/or dental hypersensitivity.

The methods of the fourth aspect comprise applying any of the compositions as described herein to the teeth, e.g., by brushing, gargling or rinsing, or otherwise administering the compositions to the oral cavity of a subject in need thereof. The compositions can be administered regularly, such as, for example, one or more times per day (e.g., twice per day). In various embodiments, administering the compositions of the present disclosure to teeth may provide one or more of the following specific benefits: (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing and/or malodor producing bacteria, (viii) treat, relieve or reduce dry mouth, (ix) clean the teeth and oral cavity, (x) whiten the teeth, (xi) reduce tartar build-up, (xii) reduce or prevent oral malodor, and/or (xiii) promote systemic health, including cardiovascular health, e.g., by reducing potential for systemic infection via the oral tissues.

The oral care compositions may be adapted for use in oral care, oral hygiene, and/or oral appearance, or for which the intended method of use comprises application to the oral cavity, and refers to compositions that are palatable and safe for topical application to the oral cavity, and for providing a benefit to the teeth and/or oral cavity. The term “oral care composition” thus specifically excludes compositions which are highly toxic, unpalatable, or otherwise unsuitable for application to the oral cavity. In some embodiments, an oral care composition is not intentionally swallowed, but is rather retained in the oral cavity for a time sufficient to affect the intended utility. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

A dual component toothpaste may be packaged in a tube containing two parallel compartments exiting via a common nozzle such that when the user extrudes the toothpaste from the package the two components mix immediately prior to application to the oral cavity. Likewise, a dual component mouthwash is typically packaged in a bottle comprising two compartments such that a measured amount of the liquid from each compartment is dispensed and mixed when the user. Dual component compositions are often used to maintain in separate components and compartments ingredients which are mutually incompatible, such that if kept in the same component they would adversely react or interfere with each other.

In contrast, a dual-phase composition, such as a mouthwash, is a single-component composition comprising two immiscible liquids which settle into two phases on standing. Such a composition has no need for separated compartments for storage because the natural tendency of the two phases to separate helps ensure that the ingredients in one phase are not maintained in intimate contact with the ingredients of the other phase. Nevertheless, when vigorously mixed, the two phases become intimately combined (such as, to form an emulsion), which may or may not separate back into the two phases on standing.

The oral care compositions of the disclosure, e.g., any of Composition 1.0 et seq or Composition 2.0 et seq, can comprise an anionic surfactant that is not sodium lauryl sulfate. For example, in one aspect, any of Composition 1.0 et seq can additionally comprise any of the following surfactants:
i. water-soluble salts of higher fatty acid monoglyceride monosulfates, such as the sodium salt of the monosulfated monoglyceride of hydrogenated coconut oil fatty acids, sodium cocomonoglyceride sulfate,
ii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate (CH₃(CH₂)₁₀CH₂(OCH₂CH₂)₂OSO₃Na),
iii. higher alkyl aryl sulfonates such as sodium dodecyl benzene sulfonate (sodium lauryl benzene sulfonate),
iv. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

By “higher alkyl” is meant, e.g., C₆₋₃₀ alkyl. In certain embodiments, the anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauroyl sarcosinate and sodium coconut monoglyceride sulfonates are examples of anionic surfactants of this type. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. In one embodiment, the anionic surfactant is present in a toothpaste at from 0.3% to 4.5% by weight, e.g., about 1.5%. The compositions of the disclosure may optionally contain mixtures of surfactants, e.g., comprising anionic surfactants and other surfactants that may be anionic, cationic, zwitterionic or nonionic. Generally, suitable surfactants are those which are reasonably stable throughout a wide pH range. Surfactants are described more fully, for example, in U.S. Pat. No. 3,959,458, to Agricola et al.; U.S. Pat. No. 3,937,807, to Haefele; and U.S. Pat. No. 4,051,234, to Gieske et al, the disclosures of which are incorporated herein by reference in their entireties.

As used herein, “nonionic surfactant” generally refers to compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name PLURONIC®), polyoxyethylene, polyoxyethylene sorbitan esters (sold under trade name TWEENS®), Polyoxyl 40 hydrogenated castor oil, fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, alkyl polyglycosides (for example, fatty alcohol ethers of polyglycosides, such as fatty alcohol ethers of polyglucosides, e.g., decyl, lauryl, capryl, caprylyl, myristyl, stearyl and other ethers of glucose and polyglucoside polymers, including mixed ethers such as capryl/caprylyl (C₈₋₁₀) glucoside, coco (C₈₋₁₆) glucoside, and lauryl (C₁₂₋₁₆) glucoside), long chain tertiary amine oxides, long chain tertiary phosphine oxides, long chain dialkyl sulfoxides, and mixtures of such materials.

In some embodiments, the nonionic surfactant comprises amine oxides, fatty acid amides, ethoxylated fatty alcohols, block copolymers of polyethylene glycol and polypropylene glycol, glycerol alkyl esters, polyoxyethytene glycol octylphenol ethers, sorbitan alkyl esters, polyoxyethylene glycol sorbitan alkyl esters, and mixtures thereof. Examples of amine oxides include, but are not limited to, laurylamidopropyl dimethylamine oxide, myristylamidopropyl dimethylamine oxide, and mixtures thereof. Examples of fatty acid amides include, but are not limited to, cocomonoethanolamide, lauramide monoethanolamide, cocodiethanolamide, and mixtures thereof. In certain embodiments, the nonionic surfactant is a combination of an amine oxide and a fatty acid amide. In certain embodiments, the amine oxide is a mixture of laurylamidopropyl dimethylamine oxide and myristylamidopropyl dimethylamine oxide. In certain embodiments, the nonionic surfactant is a combination of lauryl/myristylamidopropyl dimethylamine oxide and cocomonoethanolamide. In certain embodiments, the nonionic surfactant is present in an amount of 0.01 to 5.0%, 0.1 to 2.0%, 0.1 to 0.6%, 0.2 to 0.4%, about 0.2%, or about 0.5%, by weight based on the total weight of the oral care composition.

Mouthwashes frequently contain significant levels of ethanol, which is often needed to solubilize essential oils and to prevent bacterial contamination. High levels of ethanol may be undesirable, because in addition to the potential for abuse by ingestion, the ethanol may exacerbate conditions like xerostomia. Accordingly, in some embodiments, the oral care compositions of the invention are substantially free of ethanol, e.g., contain less than 1% ethanol, by weight based on the total weight of the oral care composition.

Humectants can enhance the viscosity, mouthfeel, and sweetness of the product, and may also help preserve the product from degradation or microbial contamination. Suitable humectants include edible polyhydric alcohols such as glycerin, sorbitol, xylitol, propylene glycol as well as other polyols and mixtures of these humectants. Sorbitol may in some cases be provided as a hydrogenated starch hydrolysate in syrup form, which comprises primarily sorbitol (the product if the starch were completely hydrolyzed to glucose, then hydrogenated), but due to incomplete hydrolysis and/or presence of saccharides other than glucose, may also include other sugar alcohols such mannitol, maltitol, and longer chain hydrogenated saccharides, and these other sugar alcohols also function as humectants in this case. In some embodiments, humectants are present at levels of 5% to 25%, e.g., 15% to 20% by weight, based on the total weight of the oral care composition.

Flavorings for use in the present invention may include extracts or oils from flavorful plants such as peppermint, spearmint, cinnamon, wintergreen, and combinations thereof, cooling agents such as menthol, methyl salicylate, and commercially available products, as well as sweeteners, which may include polyols (which also function as humectants), saccharin, acesulfame, aspartame, neotame, stevia and sucralose.

Other ingredients which may optionally be included in compositions according to the present invention include hyaluronic acid, green tea, ginger, sea salt, coconut oil, turmeric, white turmeric (white curcumin), grape seed oil, ginseng, monk fruit, vitamin E, basil, chamomile, pomegranate, aloe vera, and charcoal. Any of such ingredients may be present in an amount from 0.01% to 2% by weight of the composition, e.g., 0.01 to 1%, or 0.01 to 0.5%, or 0.01 to 0.1%, by weight based on the total weight of the oral care composition.

### EXAMPLES

Unless otherwise noted, the pH of all solutions described in the Examples is about 7. Unless otherwise noted, all figures for stannous ion concentration refer to soluble stannous, not total stannous (total stannous being soluble and insoluble stannous combined).

### Example 1—Dentifrice Formulations

Exemplary dentifrice compositions according to the present disclosure may be formulated as follows (quantities shown in % by weight of the composition):

| Ingredient | Amount (wt. %) |
|---|---|
| Demineralized water | Q.S. (~6.3%) |
| Polyethylene glycol (e.g., | 1%-5% (e.g., about 2%) |
| PEG 600) | |
| Xanthan Gum | 0.1%-1% (e.g., about 0.4%) |
| Stannous Fluoride | 0.1%-1% (e.g., about 0.454%) |
| Potassium Nitrate | 0.1%-2% (e.g., about 0.5%) |
| Tetrasodium | 0.5%-5% (e.g., about 1.2%) |
| pyrophosphate | |
| Sorbitol (70% in solution) | 20%-75% (e.g., about 61%) |
| Silicas | 10%-30% (e.g., about 23%) |
| Sodium methyl cocoyl | 0.5%-5% (e.g., about 2.5% or about 1%) |
| taurate | |
| Zwitterionic surfactant | from 0.1%-2% (e.g., about 1.25%) |
| (e.g., betaine) | |
| Flavors, Colors, and other | 0.5%-5% (e.g., about 2.1%) |
| Minors | |
| Hyaluronic acid | 0.005-0.1% (e.g., about 0.01) |
| Total | 100.0 |

While the disclosure has been described with respect to specific examples including presently preferred modes of carrying out the disclosure, those skilled in the art will appreciate that there are numerous variations and permutations of the above-described systems and techniques. It is to be understood that other embodiments may be utilized and structural and functional modifications may be made without departing from the scope of the present disclosure.

What is claimed is:

1. An oral care composition comprising:
(i) stannous fluoride or stannous chloride;
(ii) nitric acid or a water-soluble nitrate salt;
(iii) a water-soluble alkali metal polyphosphate;
(iv) an effective amount of a taurate surfactant represented by Formula (1): 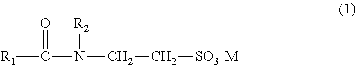
wherein R₁ is a saturated or unsaturated, straight or branched alkyl chain with 6 to 18 C atoms R₂ is H or methyl, and M⁺ is H, sodium, or potassium; and
(v) more than 10% water, by weight of the oral care composition.

2. (canceled)

3. The oral care composition of claim 1, wherein the water-soluble nitrate salt is selected from lithium nitrate, sodium nitrate, potassium nitrate, magnesium nitrate, calcium nitrate, and a combination of two or more thereof.

4. The oral care composition of claim 1, wherein the water-soluble nitrate salt is potassium nitrate.

5. The oral care composition of claim 1, wherein the water-soluble alkali metal polyphosphate is selected from a pyrophosphate, tripolyphosphate, tetraphosphate hexametaphosphate, and a combination of two or more thereof.

6. The oral care composition of claim 5, wherein the water-soluble alkali metal polyphosphate is selected from sodium pyrophosphate, potassium pyrophosphate, sodium tripolyphosphate potassium tripolyphosphate, and a combination of two or more thereof.

7. The oral care composition of claim 1, wherein the water-soluble nitrate salt is potassium nitrate and the water-soluble alkali metal polyphosphate salt is tetrasodium pyrophosphate.

8. The oral care composition of claim 1, wherein the oral care composition comprises a molar ratio of alkali metal polyphosphate to stannous fluoride or stannous chloride of 1:1 to 5:1.

9. The oral care composition of claim 1, wherein the oral care composition comprises a molar ratio of nitric acid or water-soluble nitrate salt to stannous fluoride or stannous chloride of 0.3:1 to 20:1.

10. The oral care composition of claim 1, wherein the oral care composition comprises from 0.1 to 2 wt. % stannous fluoride or stannous chloride, by weight of the oral care composition; and wherein the oral care composition comprises from 0.1 to 5 wt. % of the nitric acid or water-soluble nitrate salt, by weight of the oral care composition; and wherein the oral care composition comprises from 0.1 to 5 wt. % of the alkali metal polyphosphate salt, by weight of the oral care composition.

11. The oral care composition of claim 1, wherein the oral care composition comprises from about 50 to about 95 wt. % of water, by weight of the oral care composition.

12. (canceled)

13. The oral care composition of claim 1, wherein the oral care composition comprises from about 10 to about 50 wt. % water, by weight of the oral care composition.

14. The oral care composition of claim 1, wherein the oral care composition comprises one or more humectants in a total amount of not more than 25 wt. %, by weight of the oral care composition.

15. The oral care composition of claim 1, wherein the oral care composition is a single-phase composition.

16. The oral care composition of claim 1, wherein the oral care composition is a dual phase composition.

17. (canceled)

18. (canceled)

19. The oral care composition of claim 1, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate, sodium methyl lauroyl taurate, sodium methyl myristoyl taurate, sodium methyl oleoyl taurate, sodium methyl palmitoyl taurate, sodium methyl stearoyl taurate, and a combination of two or more thereof.

20. The oral care composition of claim 1, wherein the taurate surfactant comprises one or more surfactant selected from the group consisting of: sodium lauroyl methyl taurate, sodium methyl cocoyl taurate, and combinations thereof.

21. (canceled)

22. The oral care composition of claim 1, wherein the taurate surfactant is present in an amount of from about 0.25 to about 5 wt. %, by weight of the oral care composition.

23. (canceled)

24. The oral care composition of claim 1, wherein the oral care composition comprises hyaluronic acid or sodium hyaluronate.

25. The oral care composition of claim 1, wherein the oral care composition comprises:
stannous fluoride from about 0.1 to about 2 wt. %;
potassium nitrate from about 0.1 to about 2 wt. %;
tetrasodium pyrophosphate from about 0.1 to about 5 wt. %;
sodium methyl cocoyl taurate from about 0.25 to 5 wt. %; and
water from about 15 to about 30 wt. %, wherein all weight percentages are based on the total weight of the oral care composition.

26. The oral care composition of claim 1, wherein the oral care composition comprises:
stannous fluoride from about 0.1 to about 2 wt. %;
potassium nitrate from about 0.1 to about 2 wt. %;
sodium tripolyphosphate from about 0.1 to about 5 wt. %;
sodium methyl cocoyl taurate from about 0.25 to about 5 wt. %; and
water from about 15 to about 30 wt. %, wherein all weight percentages are based on the total weight of the oral care composition.

27-29. (canceled)
